(19) United States
(12) Patent Application Publication
(10) Pub. No.: US 2003/0166524 A1
(43) Pub. Date: Sep. 4, 2003

(54) Therapeutic uses of IL-1 receptor antagonist

(76) Inventors: John Ford, San Diego, CA (US); Alice Ho, Palo Alto, CA (US)

Correspondence Address: Li-Hsien Rin-Laures, Hyseq Inc, 670 Almanor Avenue, Sunnyvale, CA, 94085, US

(21) Appl. No.: 09/863,802

(22) Filed: May 22, 2001

Related U.S. Application Data
(63) Continuation-in-part of application No. 09/595,843, filed on Jun. 15, 2000, now abandoned.
Application No. 09/595,843 is a continuation-in-part of application No. 09/576,755, filed on May 22, 2000, now Pat. No. 6,497,870.

(51) Int. Cl. A61K 38/16

(52) U.S. Cl. 514/12

(57) ABSTRACT

The present invention provides novel therapeutic uses for interleukin-1 receptor antagonists for conditions related to interleukin-18, interleukin-12 and interferon-γ. The present invention also provides novel methods for modulating B cell proliferation.

### RELATED APPLICATIONS

This patent application is a continuation-in-part of U.S. patent application Ser. Nos. 09/595,843 filed: Jun. 16 2000 (attorney docket no. 28110/36243A) which is continuation-in-part of 09/576,755 filed May 22, 2000 (attorney docket no. 28110/36243). The above-identified applications are incorporated herein by reference in their entirety.

### FIELD OF THE INVENTION

The present invention relates to novel therapeutic uses of interleukin-1 receptor antagonist for conditions involving elevated levels of interleukin-18, interleukin-12 or interferon-γ or involving B cell or IgA disorders.

### BACKGROUND

IL-1 receptor antagonist (IL-1Ra or IRAP) is a naturally occurring protein that inhibits the activity of the proinflammatory cytokine interleukin-1 (IL-1). The IL-1 pathway consists of the two agonists IL-1α and IL-1β, a specific activation system (IL-1 converting enzyme), a receptor antagonist (IL-1Ra) produced in different isoforms and two high affinity receptors. IL-1α and IL-1β bind to two distinct IL-1 receptor types, IL-1 receptor type I (IL-1RI) and IL-1 receptor type II (IL-1RII), both of which are members of the immunoglobulin superfamily of receptors. Both types of receptors are usually coexpressed, although type I is the predominant form in fibroblasts and T cells, while type II is preferentially expressed on B cells, monocytes and neutrophils. IL-1RI and IL-1RII have different affinities for the three ligands of the IL-1 family (IL-1α, IL-1β and IL-1Ra). In particular, IL-1Ra binds to the type I receptor with an affinity similar to that of IL-1α, while IL-1Ra binds to the type II receptor 100-fold less efficiently than the type I receptor. There is evidence indicating that IL-1 induced activities are mediated exclusively via the type I receptor, whereas the type II receptor has no signaling activity and inhibits IL-1 activities by acting as a decoy for IL-1.

IL-1Ra binds to the IL-1 receptor with affinity similar to that of IL-1 but has no IL-1-like activity, even at very high concentrations, and thus inhibits (antagonizes) the activity of IL-1. The purified IL-1Ra molecule has a molecular weight of approximately 25 kD and is believed to be glycosylated. An unglycosylated recombinant form of IL-1Ra that has a molecular weight of approximately 17 kD is commercially available from R&D Systems (Minneapolis, Minn.). IL-1Ra has limited sequence similarity to IL-1α and IL-1β at the amino acid level (19% and 26%, respectively). There appear to be at least two isoforns of IL-1Ra, including a soluble form and an intracellular form generated by an alternative splicing event. IL-1Ra appears to be produced by monocytes, macrophages, neutrophils and fibroblasts; keratinocytes and cells of epithelial origin produce almost exclusively the intracellular form. In humans, the gene for IL-1Ra has been localized to the long arm of chromosome 2, which is the same region where IL-1α and IL-1β as well as IL-1RI and IL-1RII, are found.

Treatment of IL-1 related conditions through the administration of IL-1Ra molecules has been extensively studied in both in vitro and animal models. These models include those for infection, local inflammation, acute or chronic lung injury, metabolic dysfunction, autoimmune disease, immune-mediated disease, malignant disease, and host responses. In addition, human recombinant IL-1Ra has been administered to humans in clinical trials for rheumatoid arthritis, septic shock, steroid resistant graft versus host disease, acute myeloid leukemia, and chronic myelogenous leukemia. [Dinarello et al., Intern. Rev. Immunol., 16:457-499 (1998).] In these human clinical trials, IL-1Ra was not shown to significantly reduce mortality in humans with septic shock. [Fisher et al., J.A.M.A., 271:1836-43 (1994).] Clinical trials have indicated that patients tolerate administration of human recombinant IL-1Ra well without serious adverse effects.

Interleukin-18 (IL-18) is a 18.3 kD cytokine which is a strong inducer of interferon-γ (IFN-γ ). Even though IL-18 exhibits low sequence identity to the interleukin-1 (IL-1) family members (IL-1α, IL-1β, IL-1Ra), it is structurally similar to this family of cytokines. Particularly, IL-18 exhibits a 12β sheet structure that is common among IL-1 cytokine family members and shares features of the IL-1 like-signature sequences. This indicates that IL-18 is a structurally distinct cytokine compared to the IL-1 family. (See Gillespie and Horwood, Cytokine and Growth Factor Review, 9: 109-116, 1998).

The IL-18 receptor (IL-18R) was initially denoted as IL-1 receptor binding protein even though it does not bind IL-1α, IL-β, or IL-1Ra. It does however, transmit signals similar to that of IL-1R. Radiolabeled IL-18 binding studies have revealed the presence on IL-18R of high and low affinity sites for IL-18. The low affinity sites are normally available on IL-18R, while the high affinity binding sites become available when IL-18R is complexed with an accessory protein-like receptor. A soluble decoy receptor that binds IL-18 has also been identified, similar to what is observed in the IL-1 receptor system.

IL-18 is known to be expressed by activated macrophages, osteoblasts, keratinocytes, epithelial cells, pancreas cells, adrenal cells, skeletal muscle cells, liver cells, lung cells and unstimulated PBMC cells. The known functions of IL-18 include induction of IFN-γ expression in spleen cells such as T-cells, B-cells and NK cells, stimulation of T cell proliferation, enhancement of NK cell lytic cycle, enhancement of Fas ligand expression and function in T cells and NK cells, and induction of GM-CSF secretion. IL-18 also exhibits anti-viral and anti-microbial activity and suppresses tumor growth. IL-18 has also been associated with the progression of chronic inflammatory diseases including endotoxin shock, hepatitis, and autoimmune diseases such as multiple sclerosis.

The biological activities of IL-18 are exerted in synergy with interleukin-12 (IL-12). The combination of these two cytokines are known to markedly enhance production of IFN-γ in T cells and B cells. In addition to IFN-γ, the combination of IL-12 and IL-18 increases production of IL-3, IL-6 and TNF. IL-18 has also been shown to potentiate IL-12-driven Th1 cell development. Studies have indicated that IL-12 will increase expression of IL-18 receptor, which may be the mechanism for this synergy. There is evidence that this synergistic effect is carried out in vivo as well as in vitro. (See Okumura et al., Advances in Immunolgy, 70: 281-312, 1998). IL-18 and IL-12 are structurally different, bind different receptors and transduce signals through different signaling components.

IL-12 is a pro-inflammatory cytokine which was initally charcterized for its potent ability to induce production of IFNγ. IL-12 exhibits sequence homology to IL-6 and G-CSF. Unlike most cytokines, IL-12 is biologically active as a heterodimeric protein consisting of a heavy chain (p40) covalently associated with a lighter chain (p35). The cells which produce IL-12 include dentritic cells, macrophages, Langerhans cells, EBV-transformed B cells, neutrophils, keratinocytes, microglia and astrocytes.

IL-12 exhibits pleotrophic effects on multiple lymphoid cell subsets including promoting the expansion of T cells, T lymphocytes and NK-lymphokine activated killer cells. In addition, IL-12 potentiates the cytolytic activity of NK cells and cytotoxic lymphocytes. Clinically, these effects on the immune system result in enhanced host protection from infectious diseases and therefore IL-12 exhibits anti-bacterial, anit-microbial and anti-viral activity.

Antitumor activity is also induced by IL-12 enhancement of the host's natural immunity to tumorgenesis. IL-12 has also been shown to inhibit angiogenesis in tumor systems which prevents blood flow to the growing tumors. The antitumor effects are potentiated by synergism with IL-2 in vivo. Clinical studies have shown that administration of the combination of IL-12 and IL-2 significantly increases systemic production of IFNγ which leads to severe toxicity in the patient, resulting in shock and mortality.

The pro-inflammatory effects of IL-12 promote autoimmune diseases such as multiple sclerosis and arthritis. In addition, IL-12 promotes transplant rejection.

Mature B cells are derived from the bone marrow precursor cells and make up about 10-15% of the peripheral blood lymphocytes, 50% of the splenic lymphocytes and about 10% of the bone marrow lymphocytes. The primary function of the B cells is to produce antibodies. B cell development, differentiation and proliferation is regulated by cytokines. In particular, it is known that IL-7 drives pro-B and pre-pre B cell proliferation and differentiation. BCG-F (low-molecular weight B cell growth factor) and IL-1 induce B cell precursor proliferation. IL-1, IL-2, IL-4, IL-5 and IL-6 are known induce mature B cell proliferation and to drive differentiation into antibody secreting cells.

There exists a need in the art for new methods of treating conditions involving elevated levels or activity of IL-12, IL-18 and IFNγ. There also exists a need in the art for new methods of treating B cell and IgA related disorders.

### SUMMARY OF THE INVENTION

The present invention provides novel methods of using compounds that inhibit or antagonize IL-1 receptor type I, preferably IL-1Ra of SEQ ID NO: 1 or an active variant thereof, for treating a human suffering from an IL-18, IL-12, or IFN-γ related disorder or B cell and IgA related disorders i.e., a disorder resulting from or exacerbated by elevated levels of or enhanced activity of IL-18, IL-1, IFN-γ or B cells or increased levels of IgA. Specifically excluded from the definition of such IL-18, IL-12 or IFN-γ disorders are conditions for which the contemplated dosage of IL-1Ra has already been demonstrated to be an effective treatment in humans.

According to one aspect of the invention, the novel methods of treatment comprise administering to a human an IL-1Ra polypeptide comprising the amino acid sequence of SEQ ID NO: 1, or a variant thereof that retains biological activity, in an amount effective to inhibit (either partially or completely) the activity of IL-18 and/or IL-12 in the human subject. Beneficial effects may even be provided by a dosage that provides partial inhibition of IL-18 or IL-12 activity, e.g., approximately 90%, 80%, 70%, 60%, 50%, 40%, 30% or 20% inhibition compared to baseline.

The methods optionally further include a step of measuring IL-18, IL-12 and/or IFN-γ levels or activity in a tissue or fluid sample from the subject. Levels of or activity (e.g., proinflammatory activity) of IL-18 and/or IL-12 can be measured in any way known in the art, including by measuring circulating or local levels of IFNγ, NK cell activation, or serum IgE levels.

Another aspect of the present invention provides a method of treating a human diagnosed with an IL-18, IL-12 or IFN-γ related disorder by measuring circulating or local levels or activity of IL-12, IL-18, and/or IFN-γ in a tissue or fluid sample from the human subject, followed by treating with a therapeutically effective amount of a compound that antagonizes or inhibits IL-1R, preferably IL-1Ra or an active variant thereof. Such measurements may be carried out before or concurrently with the IL-1Ra treatment.

Yet a further aspect of the invention provides a method of monitoring IL-1R antagonist/inhibitor treatment of a human with an IL-18, IL-12 or IFN-γ related disorder by measuring circulating or local levels or activity of IL-12, IL-18, or IFN-γ in a tissue or fluid sample from the human subject. Such measurements may be carried out before, concurrently and/or after administering the therapeutically effective amount of IL-1R antagonist/inhibitor.

These novel therapeutic uses of IL-1Ra are specifically contemplated for IL-18 related disorders including endotoxin induced liver injury, hepatitis, multiple sclerosis, and haemophagocytic lymphocytosis. The therapeutic uses of IL-1Ra are also specifically contemplated for IL-12 related disorders including multiple sclerosis and IL-12 induced cytotoxicity resulting from antitumor therapy.

The invention also includes compounds for the preparation of medicaments useful for treating IL-12, IL-18 and/or INF-γ related disorders. It is contemplated that therapeutic methods according to the invention include treating IL-12 or IL-18 related disorders by concurrent administration of IL-1Ra, or an active variant thereof, and a second therapeutic agent. The second agent may be another IL-1β antagonist such as antibody to IL-1R type I or IL-1β antibody, or another antagonist to IL-18 such as IL-18R antibody or IL-18 antibody.

Another aspect of the invention provides a method of treating any inflammatory disease state mediated by IL-18 by administering to a subject in need thereof an amount of IL-1Ra or an active variant thereof effective to inhibit IL-18 activity. Also provided are in vitro as well as in vivo methods of inhibiting IL-18 activity.

Also contemplated is the use of compounds that inhibit or antagonize IL-1R type I, preferably IL-1Ra, or analogs thereof, in the preparation of a medicament for the treatment of IL-18 and/or IL-12 related disorders.

Also provided are compositions that comprise a dosage of IL-1Ra or an analog thereof, that is at least 10-fold less than the dosage required to completely inhibit IL-1 induced PGE₂ production. Preferable dosages of IL-1Ra are at least 100-fold, 500-fold, 1000-fold, 5000-fold, or 10,000-fold less than the dosage required to completely inhibit PGE₂ production.

The invention also provides for methods of inhibiting B cell proliferation comprising administering an inhibitor of interleukin-1 receptor antagonist activity to human with elevated B cell levels or B cell activity. Optionally, before, concurrently or after administration, B cell levels or activity may be measured in said human. The inhibitor of IL-1Ra activity may be for example, an antibody to IL-1Ra, an antisense oligonucleotide, an inactive variant of IL-1Ra or a soluble form or a receptor that binds to IL-1Ra, or a small molecule that inhibits binding of or activity of IL-1Ra activity. The methods of inhibiting B cell proliferation can be an effective therapy for B cell related disorders such as B cell lymphoproliferative disorders (e.g., myelomas, lymphomas, leukemias) and B cell related autoimmune diseases. The invention also includes compounds for the preparation of medicaments useful for the inhibition of B cell proliferation in a human suffering from a B cell related disorder.

The invention also provides for methods of stimulating B cell proliferation comprising administering an effective amount of interleukin-1 receptor antagonist, comprising the amino acid sequence of SEQ ID NO: 1 or a variant thereof, to a human suffering from a B cell immune deficiency or otherwise in need of higher B cell levels or activity (e.g., suffering from infection). Optionally, before, concurrently or after administration, B cell or antibody levels in said human may be measured. The invention also includes compounds for the preparation of medicaments useful for the simulation of B cell proliferation in a human with B cell related disorders.

Also encompassed by the invention are methods for treating autoimmune diseases associated with increased production of IgA. These methods comprise administering an effective amount of IL-1Ra to a human suffering from a disorder related to elevated IgA levels. These disorder include but are not limited to IgA nephropathy, dermatitis herpetiformis and linear IgA disease. Optionally, before, concurrently or after the administration, IgA levels may be measured in said human.

Numerous additional aspects and advantages of the invention will become apparent to those skilled in the art upon consideration of the following detailed description of the invention which describes presently preferred embodiments thereof.

### BRIEF DESCRIPTION OF DRAWINGS

FIG. 1 displays the inhibition of IL-18 induced IFNγ production by varying dosages of IL-1Ra. PBMC cells were treated with 100 ng/ml IL-18 in the presence of 0.5 μg/ml anti-CD3 antibody for 36 hours. The cells were treated with different molar fold excess (0.0001-fold to 100-fold) of IL-1Ra in the presence of IL-18. The amount of IFNγ was measured by ELISA.

FIG. 2 displays IL-18 stimulation of IL-1 production in PBMC cells. The cells were treated with 100 ng/ml IL-18 in the presence of 0.5 μg/ml anti-CD3 antibody for 36 hours. The amount of IL-1β was measured by ELISA.

### DETAILED DESCRIPTION OF THE INVENTION

The present invention provides novel therapeutic uses for compounds that inhibit or antagonize IL-1 receptor type I, preferably interleukin-1 receptor antagonist (IL-1Ra) or active variants thereof Other compounds that inhibit or antagonize IL-1 receptor type I include: antibody specific for IL-1 receptor type I, antibody specific for IL-1 receptor accessory protein, IL-1Hy1 (described in co-owned, concurrently filed U.S. patent application Ser. No. 09/576,008 [Attorney Docket No. 28110/36456] and in prior related Int'l Application No. PCT/US99/04291 filed Apr. 5, 1999 (Int'l Publication No. WO 99/51744), the disclosures of all of which are incorporated by reference, and IL-1Hy2 (described in co-owned, concurrently filed U.S. patent application Ser. No. 09/578,458 [Attorney Docket No. 28110/36479] and co-owned, concurrently filed Int'l Application No. PCT US00/14144 filed May 22, 2000 (Int'l Publication No. WO) [Attorney Docket No. 28110/36479/PCT], the disclosures of all of which are incorporated by reference.

The therapeutic uses as described herein with respect to IL-1Ra and active variants thereof encompass disorders associated with IL-12, IL-18, or IFN-γ or B cell proliferation or activity (including B cell development, differentiation, maturation, proliferation, or activation, both antigen-independent and antigen dependent) or disorders related to elevated levels of IgA that are not involved in disease states wherein IL-1 receptor antagonist is known to be an effective treatment in humans, e.g., rheumatoid arthritis. In contrast, IL-1Ra has not been shown to be effective for treating septic shock. Human recombinant IL-1Ra has been administered to humans in clinical trials for rheumatoid arthritis, septic shock, steroid resistant graft-versus-host disease, acute myeloid leukemia, and chronic myelogenous leukemia. IL-1Ra has been tested in some animal models of disease including models for infection, local inflammation, acute or chronic lung injury, metabolic dysfunction, autoimmune disease, immune-mediated disease, malignant disease, and host responses.

Therapeutic uses as described herein encompass any and all IL-18 or IL-12 related/mediated disorders or B cell or IgA related disorders wherein the dosage of IL-1Ra that has been shown to be therapeutically effective in humans is different from the dosage contemplated herein (i.e., a dosage that provides partial or complete inhibition of IL-18 and/or IL-12 and/or IFN-γ activity). A relatively low dosage of IL-1Ra is expected to be effective for blocking IL-18 induced IFN-γ production as described herein. Specifically contemplated for treating rheumatoid arthritis are doses of IL-1Ra less than 70 mg/day, preferably less than 30 mg/day.

The present invention also provides methods for identification or diagnosis of patients suffering from IL-18 and/or IL-12 related disorders for whom IL-1Ra treatment would be suitable, as well as methods for monitoring IL-1Ra treatment of patients. Such methods, which involve measuring IL-18, IL-12 and/or IFN-γ activity in a tissue or fluid sample from the human subject, are contemplated for any and all disorders associated with IL-12, IL-18 or IFN-γ.

IL-18 Related Disorders

IL-18 has been found to have a variety of biological activities including the stimulation of activated T cell proliferation, enhancement of NK cell lytic activity, induction of IFNγ secretion, enhancement of Fas ligand expression and function, and stimulation of granulocyte-macrophage colony-stimulating factor (GM-CSF) production by activated T cells. IL-18 has been shown to counteract viral and intracellular infections and suppress tumor formation. However, IL-18 is also involved in the pathogenic progression of chronic inflammatory diseases, including endotoxin-induced shock, liver injury (including endotoxin-induced liver injury, hepatitis, biliary atresia and obesity-related fatty liver) and autoimmune diseases such as multiple sclerosis. Other disorders related to IL-18 production include meliodosis, purine nucleoside phosphorylase deficiency, increased susceptibility to Leishmania major and Staphylococcus aureus infection, hemophagocytic lymphohistiocytosis, mononucleosis, viral meningitis/encephalitis, bacterial meningitis/encephalitis and ischemia or ischemia/reperfusion injury.

Inflammation may result from infection with pathogenic organisms (including gram-positive bacteria, gram-negative bacteria, viruses, flingi, and parasites such as protozoa and helminths), transplant rejection (including rejection of solid organs such as kidney, liver, heart, lung or cornea, as well as rejection of bone marrow transplants including graft versus host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, some forms of diabetes, or any other autoimmune state where attack by the subject's own immune system results in pathologic tissue destruction. Allergic reactions include allergic asthma, chronic bronchitis, allergic rhinitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

The development of multiple sclerosis (MS) is an example of an inflammatory autoimmune disease that involves IL-18 and IFN-γ. In the experimental allergic encephalomyelitis animal model (EAE), the demyelinating effect on the central nervous system is similar to that in humans suffering from MS. Administration of IL-18 neutralizing antibodies have been reported to block development of EAE in rats. (Wildbaum et al., J. Immunol. 161(11): 6368-74, 1998).

Endotoxin Related Diseases

Endotoxin activation of the systemic inflammatory response leads to a number of disorders including bacterial and/or endotoxin-related shock, fever, tachycardia, tachypnea, cytokine overstimulation, increased vascular permeability, hypotension, complement activation, disseminated intravascular coagulation, anemia, thrombocytopenia, leukopenia, pulmonary edema, adult respiratory distress syndrome, intestinal ischemia, renal insufficiency and failure, and metabolic acidosis

Liver Injury

Hepatitis represents liver disorders that are characterized by hepatic inflammation and necrosis that can be manifested as an acute or chronic condition. These liver disorders include virus-induced hepatitis such as hepatitis A, hepatitis B, hepatitis C (non-A, non-B hepatitis), hepatitis D, hepatitis E; toxin and drug induced hepatitis such as acetaminophohen hepatotoxicity, halothane hepatotoxicity, mehtyldopa hepatoxicity, iaoniazid hepatoxicity, sodium valproate hepatoxicity, phenytion hepatoxicity, chlorpromazine hepatoxicity, amiodarone hepatoxicity, amioidarone hepatoxicity, erythromycin hepatoxicity, oral contraceptive hepatoxicity, 17,α-alkyl-substituted anabolic steroid hepatoxicity and trimethoprim-sulfamethoxazole hepatoxicity; cholestatic hepatitis; alcoholic hepatitis; autoimmune chronic active hepatitis; and T cell mediated hepatitis. Other conditions that cause liver injury include congenital bilary atresia, obesity-related fatty liver and the autosomal recessive disease heamophagocytic lymphohistocytosis (HLH).

IL-18 induced IFN-γ plays a role in liver injury. IFNγ has been shown to mediate LPS-induced liver injury following Propionibacterium acnes infection as described in Tsuji et al. (J. Immunol. 162: 1049-55, 1999). Large number of macrophages and lymphocytes infiltrate the portal area in response to P. acnes infection which results in intrahepatic formation of granulomas. IFNγ knock out mice exhibited less macrophage infiltration and a reduction in the number and size of granulomas. Subsequent treatment with low doses of LPS caused massive hepatic necrosis and increased IL-12, IL-18 and TNF-α serum levels in the normal mice, while the knock out mice exhibited drastic decreases in IL-12, IL-18 and TNF-α serum levels. The addition of IFNγ neutralizing antibody also caused a decrease in IL-18 and IL-12 levels. This model of liver injury indicates that LPS-induced liver injury is associated with increased levels of IL-18, IL-12 and IFN-γ. Currently, a role for IL-1 is not known in this liver injury model. Since IL-1β is known to be induced by LPS, it is possible IL-1β also plays a role in the disorder. Treatment with IL-1Ra may modulate the severity of liver injury due to IL-18 induced IFN-γ production and IL-1β.

IL-18 has also been shown to be involved in the immunomediated hepatitis model where treatment with concavalin A induced hepatitis in mice as described by Fiorucci et al. (Gastroenterology 118: 404-21, 2000). In this model, CD+Tcells and Th1-like cytokines cause Fas mediated liver cell death. Treatment with a nitric oxide derivative of aspirin protected against this cell death by reducing production of IFNγ, IL-18, IL-12, IL-1β and TNF-α. In addition, a neutralizing antibody to IL-18 caused a decrease in IFNγ production and reduced liver injury induced by conA.

HLH is a fatal autosomal recessive disease that manifests in early childhood. This disease is characterized by fever, hepatosplenomegaly, cytopenia and widespread infiltration of vital organs by activated lymphocytes and macrophages. Patients with HLH exhibit elevated serum levels of IL-18. IL-18 plays an important role in the induction of Th1 cells in HLH patients. (Takada et al., Br. J. Haematol. 106: 182-9, 1999).

IL-1Ra inhibits IL-18 induced production of IFNγ. In the models described above, the degree of IL-1β activity is not known. Since IL-1β is known to be induced by LPS, it is possible that IL-1β also play a role in the pathogenicity of these conditions. The presence of the appropriate amount of IL-1Ra may modulate the severity of the disease states due to both IL-18 induced IFNγ production and IL-1β.

IL-12 Related Disorders

IL-12 is known to potentiate IFNγ production, and the cytolytic activity of NK cells and cytotoxic T lymphocytes. These immunomodulatory effects have implicated a role for IL-12 in therapies for cancer and infectious disease. However, these same therapeutic effects can also promote autoimmune diseases and chronic inflammatory conditions such as multiple sclerosis, transplant rejection and cytotoxicity.

IL-12 and IFN-γ are involved in the pathogenesis of multiple sclerosis (MS). In the experimental allergic encephalomyelitis animal model (EAE), the demyelinating effect on the central nervous system is carried out similar to that in humans suffering from MS. Currently, IFNβ is used to treat MS. The mechanism of IFNβ treatment may be to decrease the number of IFNγ producing T cells in MS patients. (Rep et al., J. Neuroimmunol. 96:92-100, 1999). In addition, IFNγ production in blood lymphocytes was found to correlate with disability score in MS patients. (Petcreit et al., Mult. Scler. 6: 19-23, 2000). Antibodies against IL-12 were found to prevent superantigen-induced and spontaneous relapses of EAE in mice (Constantineseu et al., J. Immunol. 161: 5097-5104, 1998). All these studies point to the involvement of IL-12 induced IFNγ production in the progression of MS in human patients. Therefore, IL-1Ra treatment to reduce IFNγ production may be a useful therapy for MS patients.

The combination of IL-12 and IL-2 has synergistic anti-tumor activity in vivo. However, in clinical trials the combination resulted in significant toxicity and subsequently shock and mortality. (Cohen, Science 270: 908 1995). In a murine model investigated by Carson et al. (J. Immunol., 162: 4943-5, 1999) determined that the fatal systemic inflammatory response was NK cell dependent but not related to other effector molecules in the system such as IL-1, TNF-α, and IFNγ. Since IL-1Ra inhibits IL-12 induced IFN-γ production is expected to inhibit other biological activities of IL-12 such as NK cell cytolytic activity. Inhibition of NK cell activity, through IL-1Ra administration, may reduce toxicity resulting from IL-12 antitumor treatment.

B Cell Related Disorders

Mature B cells are derived from the bone marrow precursor cells and make up about 10-15% of the peripheral blood lymphocytes, 50% of the splenic lymphocytes and about 10% of the bone marrow lymphocytes. The primary function of the B cells is to produce antibodies. B cell development, differentiation and proliferation is regulated by cytokines. In particular, it is known that IL-7 drives pro-B and pre-pre B cell proliferation and differentiation. BCG-F (low-molecular weight B cell growth factor) and IL-I induce B cell precursor proliferation. IL-1, IL-2, IL-4, IL-5 and IL-6 are known induce mature B cell proliferation and to drive differentiation into antibody secreting cells. In Example 7, it is demonstrated that mature B cell proliferation was stimulated by IL-1Ra.

Leukemias can result from uncontrolled B cell proliferation initially within the bone marrow before disseminating to the peripheral blood, spleen, lymph nodes and finally to other tissues. Uncontrolled B cell proliferation also may result in the development of lymphomas which arise within the lymph nodes and then spread to the blood and bone marrow. Inhibition of B cell proliferation may be effective in treating leukemias, lymphomas and myelomas including but not limited to multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma, B cell chronic lymphocytic leukemia, acute lymphoblastic leukemia, and hairy cell leukemia. Uncontrolled B cell proliferation is also associated with other lymphoproliferative diseases such as multicentric Castleman's disease, primary amyloidosis, Franklin's disease, Seligrnann's disease and primary effuision lymphoma.

Autoimmune diseases can be associated with hyperactive B cell activity which results in autoantibody production. Inhibition of the proliferation of activated mature B cells proliferation may be therapeutically effective in decreasing the levels of autoantibodies in autoimmune diseases including but not limited to systemic lupus erythematosus, Crohn's Disease, graft-verses-host disease, Graves' disease, myasthenia gravis, autoimmune hemolytic anemia, autoimmune thrombocytopenia, asthma, cryoglubulinemia, primary biliary schlerosis, pernicious anemia, Waldenstrom macroglobulinemia, hyperviscosity syndrome, macroglobulinemia, cold agglutinin disease, monoclonal gammopathy of undetermined origin, anetoderma and POEMS syndrome (polyneuropathy, organomegaly, endocrinopathy, M component, skin changes).

The stimulation of B cell proliferation may be desired to treat immune deficiency disorders which are associated with reduced levels of circulating antibodies or other conditions, such as infection, wherein increased B cell activity is desirable. Administration of IL-1Ra to stimulate the proliferation of B cell populations would be useful for the treatment of immune deficiency disorders including but not limited to severe combined immunodefiency syndrome (SCID), adenosine deaminase (ADA) deficiency, purine nucleoside phosphorylase (PNP) deficiency, MHC class II deficiency, immunodefeciency with thymona, and reticular dysgenesis. Administration of IL-1Ra to stimulate B cell proliferation would also be useful in treating immunoglobulin deficiency syndromes including but not limited to agammagglobulinemia, transient hypogammaglobulinemia of infancy, isolated deficiency of IgG, common variable immunodeficiency, X-linked immunodeficiency with increased levels of IgM and isolated deficiency of IgM. Infectious diseases include viral, bacterial, fungal, protozoan and parasitic infections.

As described briefly above, B cell related disorders may either have increased levels of B cells (e.g. leukemias), hyperactivated B cells (e.g. autoimmune diseases) or decreased levels of B cells (e.g. immune deficiency syndromes). As described in Example 7, IL-1Ra significantly simulates B cell proliferation. Therefore, IL-1Ra may be administered to stimulate B cell proliferation in immune deficiency syndromes while inhibition of IL-1Ra activity may be useful in treatment of B cell hyperproliferative disorders such as leukemia and autoimmune diseases.

Measure of Effectiveness of IL-1Ra Treatment

The effect of IL-1 Ra on IL-12 and/or IL-18 activity in the disorder may be determined by measuring the biological activities of these cytokines. Both IL-12 and IL-18 are known to induce IFNγ production in T cells. In addition to IFN-γ, the combination of IL-12 and IL-18 increases production of IL-3, IL-6 and TNF. Treatment with IL-1Ra is expected to reduce IFNγ production induced by IL-12 and IL-18. Circulating or local levels of IFNγ in tissue or fluid samples from patients treated with IL-1Ra will be an indication of the therapeutic effects of IL-1Ra on the IL-18 and IL-12 related disorders. Tissue samples include tissue samples from an area involved in inflammation or other disease. Fluid samples include, for example, whole blood, plasma, serum, cerebrospinal fluid, synovial fluid, peritoneal fluids (including lavage fluids or exudate), pleural fluids (including lavage fluids or exudate), wound fluids (including lavage fluids or exudate).

Furthermore, IL-12 is known to activate NK cells and to decrease serum IgE levels. These assays may also be used to measure the effectiveness of IL-1Ra treatment for IL-12 related disorders. The NK cell cytolytic activity in patients treated with IL-1Ra can be assayed by measuring patient's blood samples ability to lyse colon carcinoma or lymphoma cells in vitro. (Lieberman et al., J. Sur. Res., 50: 410-415, 1992) In addition, the serum levels of IgE from patients treated with IL-1Ra can be measured to determine the effectiveness of treatment for IL-12 related disorders. (Kiniwa et al. J. Clin. Invest., 90: 262-66, 1992) To treat the IL-18 IL-12 related disorders, IL-1Ra will be administered to patients suffering from said disorders in an amount effective to inhibit the activity of IL-18 and/or IL-12. As used herein, the term “IL-1 receptor antagonist (IL-1Ra)” refers to any polypeptide comprising the amino acid sequence of SEQ ID NO: 1 or an active variant thereof.

To treat B cell related disorders, IL-1Ra polypeptide or an inhibitor of IL-1Ra activity will be administered to patient suffering from said disorder in an effective amount to either stimulate or inhibit B cell proliferation. B cell proliferation can be measured by quantitating the level of mature B cells within in a fluid or tissue sample of the treated patient including fluid samples such as blood, plasma, serum, lymphatic fluid samples and tissue samples including bone marrow and spleen samples. B cell proliferation can also be measured indirectly by measuring the level of antibodies within in a fluid or tissue sample of the treated patient.

To treat IgA related disorders, IL-1Ra polypeptide or an inhibitor of IL-1Ra activity will be administered to patient suffering from said disorder in an effective amount to either inhibit IgA production. IgA production can be measured by quantitating the IgA levels within in a fluid or tissue sample of the treated patient including fluid samples such as blood, plasma, serum, secretions and lymphatic fluid samples and tissue samples including bone marrow, spleen, or skin samples.

The term “variant” (or “analog”) as used herein refers to any polypeptide differing from naturally occurring polypeptides by amino acid insertions, deletions, and substitutions, created using, e g., recombinant DNA techniques. Variants that comprise amino acid sequence having at least about 70%, about 75%, about 80%, about 85%, about 90%, about 95%, or higher sequence identity to SEQ ID NO: 1, and that retain the desired biological activity of SEQ ID NO: 1, are contemplated in the uses according to the present invention. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence. Guidance can also be provided by various three-dimensional protein modeling programs known in the art. In general, conservative substitutions are expected to provide a variant that retains biological activity of wild type polypeptide.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

The present invention provides for the use of isolated polypeptides encoded by the nucleic acid fragments of the IL-1Ra polynucleotide sequence or by degenerate variants of the nucleic acid fragments of the IL-1Ra polynucletoide. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins. A variety of methodologies known in the art can be utilized to obtain any one of the isolated polypeptides or proteins of the present invention. At the simplest level, the amino acid sequence can be synthesized using commercially available peptide synthesizers. This is particularly useful in producing small peptides and fragments of larger polypeptides. Fragments are useful, for example, in generating antibodies against the native polypeptide. In an alternative method, the polypeptide or protein is purified from host cells which produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments encoding greater than about 100 amino acids, or greater than about 200 amino acids, and fragments that encode specific protein domains.

Other fragments and derivatives of the IL-1Ra which would be expected to retain IL-1Ra antagonistic activity in whole or in part and may thus be useful for screening or other immunological methodologies may also be easily made by those skilled in the art given the disclosures herein. Regions of the protein that are important for its function can be determined by various methods known in the art including the alanine-scanning method which involves systematic substitution of each amino acid residue by alanine, followed by testing of the alanine-substituted variants of the protein for receptor activity. This type of analysis determines the importance of the substituted amino acid residue for activity.

The IL-1Ra polypeptides of the invention include IL-1Ra analogs (variants). This embraces fragments of IL-1Ra, as well as analogs (variants) thereof in which one or more amino acids has been deleted, inserted, or substituted. Analogs of the invention also embrace fusions or modifications of IL-1Ra wherein the protein or analog is fused to another moiety or moieties, e.g., targeting moiety or another therapeutic agent. Such analogs may exhibit improved properties such as activity and/or stability.

The IL-1Ra fragments may also be fused to carrier molecules such as immunoglobulins or fragments thereof for many purposes, including increasing half life or the valency of protein binding sites. For example, fragments of the protein may be fused through “linker” sequences to the Fc portion of an immunoglobulin. For a bivalent form of the protein, such a fusion could be to the Fc portion of an IgG molecule. Other inmmunoglobulin isotypes may also be used to generate such fusions. For example, a protein-IgM fusion would generate a decavalent form of the protein of the invention.

Variants also include IL-1Ra polypeptides chemically modified by such techniques as ubiquitination, labeling (e.g., with radionuclides or various enzymes), pegylation (derivatization with polyethylene glycol) and insertion or substitution by chemical synthesis of amino acids such as omithine, which do not normally occur in human proteins.

Antibodies

Another aspect of the invention is an antibody that specifically binds the IL-1Ra polypeptide. Such antibodies include monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR and/or antigen-binding sequences, which specifically recognize a polypeptide of the invention. Preferred antibodies of the invention are human antibodies which are produced and identified according to methods described in WO93/11236, published Jun. 20, 1993, which is incorporated herein by reference in its entirety. Antibody fragments, including Fab, Fab′, F(ab′)₂, and Fᵥ, are also provided by the invention. The term “specific for” indicates that the variable regions of the antibodies of the invention recognize and bind IL-1Ra polypeptides exclusively (i.e., able to distinguish a IL-1Ra polypeptide from other related interleukin family members despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the IL-1Ra polypeptides of the invention are also contemplated, provided that the antibodies are first and foremost specific for, as defined above, IL-1Ra polypeptides. As with antibodies that are specific for full length polypeptides, antibodies of the invention that recognize IL-1Ra fragments are those which can distinguish IL-1Ra polypeptides from the family of polypeptides despite inherent sequence identity, homology, or similarity found in the family of proteins. Antibodies of the invention can be produced using any method well known and routinely practiced in the art.

Non-human antibodies may be humanized by any methods known in the art. In one method, the non-human CDRs are inserted into a human antibody or consensus antibody framework sequence. Further changes can then be introduced into the antibody framework to modulate affinity or immunogenicity.

Antibodies of the invention are useful for, for example, therapeutic purposes (by modulating activity of a polypeptide of the invention), diagnostic purposes to detect or quantitate a polypeptide of the invention, as well as purification of a polypeptide of the invention.

Proteins of the invention may also be used to immunize animals to obtain polyclonal and monoclonal antibodies which specifically react with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987). In general, techniques for preparing polyclonal and monoclonal antibodies as well as hybridomas capable of producing the desired antibody are well known in the art (Campbell, A. M., Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21 (1990); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96).

Any animal (mouse, rabbit, etc.) which is known to produce antibodies can be immunized with a peptide or polypeptide of the invention. Methods for immunization are well known in the art. Such methods include subcutaneous or intraperitoneal injection of the polypeptide. One skilled in the art will recognize that the amount of the protein encoded by the ORF of the present invention used for immunization will vary based on the animal which is immunized, the antigenicity of the peptide and the site of injection. The protein that is used as an immunogen may be modified or administered in an adjuvant in order to increase the protein's antigenicity. Methods of increasing the antigenicity of a protein are well known in the art and include, but are not limited to, coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Research. 175:109-124 (1988)). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single chain antibodies (U.S. Pat. No. 4,946,778) can be adapted to produce single chain antibodies to proteins of the present invention.

For polyclonal antibodies, antibody containing antiserum is isolated from the immunized animal and is screened for the presence of antibodies with the desired specificity using one of the above-described procedures. The present invention further provides the above-described antibodies in delectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, etc. Procedures for accomplishing such labeling are well-known in the art, for example, see (Stemberger, L. A. et al., J. Histochem. Cytochem. 18:315 (1970); Bayer, E. A. et al., Meth. Enzym. 62:308 (1979); Engval, E. et al., Immunol. 109:129 (1972); Goding, J. W. J. Immunol. Meth. 13:215 (1976)).

Pharmaceutical Formulations and Routed of Administration

Recombinant human IL-1Ra is commercially available from R& D Systems (Minneapolis, Minn.). IL-1Ra polypeptide or other composition of the present invention (from whatever source derived, including without limitation from recombinant and non-recombinant sources) may be administered to a patient in need, by itself, or in pharmaceutical compositions where it is mixed with suitable carriers or excipient(s) at doses to treat or ameliorate IL-12 and/or IL-18 and/or IFN-γ related disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1 (including IL-1β), IL-2, IL-3, IL-4, IL-5, IL-6, IL-7, IL-8, IL-9, IL-10, IL-11, IL-12, IL-13, IL-14, IL-15, IFN, G-CSF, thrombopoietin, stem cell factor, and erythropoietin. In further compositions, proteins of the invention may be combined with other agents beneficial to the treatment of IL-12 and/or IL-18 related diseases. These agents include various growth factors such as epidermal growth factor (EGF), platelet-derived growth factor (PDGF), transforming growth factors (TGF-α and TGF-β), insulin-like growth factor (IGF), as well as cytokines described herein.

The pharmaceutical composition may further contain other agents which either enhance the activity of the IL-1Ra or other active ingredient or compliment its activity or use in treatment. Such additional factors and/or agents may be included in the pharmaceutical composition to produce a synergistic effect with IL-1Ra, or to minimize side effects.

IL-1β and IL-18 may act synergistically to stimulate production of IFNγ. It may be beneficial to treat IL-18 related diseases with a composition containing, e.g., synergistic amounts of IL-1Ra and an antagonist to IL-18 such as IL-18 receptor antibody or IL-18 antibody. Further, it may be beneficial to administer compositions containing, e.g., synergistic amounts of IL-1Ra and another IL-1β antagonist such as antibody to IL-1R type I or IL-1β antibody, which would enhance the suppression of IFNγ production in IL-18 and/or IL-12 related diseases.

It is hypothesized that IL-1β and IL-18 may act synergistically or are least linked to a common biological pathway. If low concentrations of IL-1β are required to synergize with IL-18 to produce IFNγ, only low molar amounts of IL-1Ra could be required to block IL-18 induced IFNγ production. Alternatively, in addition to IL-1Ra binding to IL-1R, IL-1Ra may sterically hinder the association of IL-18 to its receptor. Since IL-1Ra is not known to bind IL-18R (Parnet et al., J. Biol. Chem. 271(8) 3967-70, 1996), it is possible that IL-18R and IL-1R may multimerize to form a receptor subunit complex that induces IFNγ production.

As an alternative to being included in a pharmaceutical composition of the invention including an IL-1Ra polypeptide, a second protein or a therapeutic agent may be concurrently administered with the IL-1Ra polypeptide (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of an IL-12 and/or IL-18 related disorder, or an increase in rate of treatment, healing, prevention or amelioration of an IL-12 and/or IL-18 related disorder. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment, a therapeutically effective amount of protein or other active ingredient of the present invention is administered to a mammal having an IL-12 and/or IL-18 related disorder to be treated. IL-1Ra polypeptide or other active ingredient of the present invention may be administered in accordance with the method of the invention either alone or in combination with other therapies such as treatments employing cytokines, lymphokines or other hematopoietic factors. When co-administered (i.e., concurrently administered) with one or more cytokines, lymphokines or other hematopoietic factors, IL-1Ra or other active ingredient of the present invention may be administered either simultaneously with the cytokine(s), lymphokine(s), other hematopoietic factor(s), thrombolytic or anti-thrombotic factors, or sequentially (i.e., before or after). If administered sequentially, the attending physician will decide on the appropriate sequence of administering IL-1Ra polypeptide or other active ingredient of the present invention in combination with cytokine(s), lymphokine(s), other hematopoietic factor(s), thrombolytic or anti-thrombotic factors.

Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of IL-1Ra or other active ingredient of the pharmaceutical composition can be carried out in a variety of conventional ways, such as oral ingestion, inhalation (e.g. in an aerosolized or nebulized formulation for delivery to the lungs), topical application or cutaneous, subcutaneous, intraperitoneal, parenteral or intravenous injection. Intravenous administration to the patient is preferred.

Alternately, one may administer the compound in a local rather than systemic manner, for example, via injection of the compound directly into an affected tissue, often in a depot or sustained release formulation. Furthermore, one may administer the drug in a targeted drug delivery system, for example, in a liposome coated with a specific antibody, targeting the affected tissue. The liposomes will be targeted to and taken up selectively by the afflicted tissue.

Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of IL-1Ra or other active ingredient of the present invention is administered orally, IL-1Ra or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% IL-1Ra polypeptide or other active ingredient of the present invention, and preferably from about 25 to 90% IL-1Ra or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of Il-1Ra polypeptide or other active ingredient of the present invention, and preferably from about 1 to 50% IL-1Ra polypeptide or other active ingredient of the present invention.

When a therapeutically effective amount of IL-1Ra polypeptide or other active ingredient of the present invention is administered by intravenous, cutaneous or subcutaneous injection, IL-1Ra polypeptide or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to IL-1Ra polypeptide or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxyrnethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counterions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface imnuunoglobulin and other molecules on B cells as well as antibodies able to bind the TCR and other molecules on T cells can be combined with the pharmaceutical composition of the invention. The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The amount of IL-1Ra or other active ingredient of the present invention in the pharmaceutical composition of the present invention will depend upon the nature and severity of the condition being treated, and on the nature of prior treatments which the patient has undergone. Ultimately, the attending physician will decide the amount of IL-1Ra or other active ingredient of the present invention with which to treat each individual patient. Initially, the attending physician will administer low doses of IL-1Ra or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of IL-1Ra or other active ingredient of the present invention per kg body weight.

Compositions of the present invention include therapeutic method administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the affected tissue. Therapeutically useful agents other than IL-1Ra or other active ingredient of the invention which may also optionally be included in the composition as described above, may alternatively or additionally, be administered simultaneously or sequentially with the composition in the methods of the invention. Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of IL-18 and/or IL-12 induced IFN-γ production). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for IL-1Ra will be in the range of about 0.01 to 100 mg/kg of body weight daily, with the preferred dose being about 0.1 to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

Previous clinical trials have determined that administration of IL-1Ra is well tolerated and does not cause serious side effects. For example, in patients suffering from rheumatoid arthritis, 30, 75, or 150 mg/day of recombinant IL-1Ra was self-administered as a single subcutaneous injection at the site of arthritis. This treatment caused a dose dependent reduction in the number of swollen joints and overall patient scores; decrease in C-reactive and sedimentation rates; and 50% reduction in new bone erosions. (See Brenihan, Ann. Rheum. Dis. 58:196-498, 1999).

In patients suffering from septic shock, IL-1Ra was administered as a loading bolus of 100 mg followed by 3 day infusion of 17, 67, or 133 mg/hr of IL-1Ra. In Phase II clinical trials, a dose dependent decrease in mortality was observed where 44% mortality in patients receiving the lowest dose and 16% mortality in group receiving the highest dose. (Fisher et al., Crit. Care Med. 22: 12-21, 1994). In further Phase III clinical trials, however, no statistically significant reduction in mortality was observed with IL-1Ra treatment.

Patients exhibiting graft-versus-host disease received 400-3400 mg/day of IL-1Ra continuously every 24 hours for 7 days as intervenous infusions. This treatment resulted in an improvement in 16 out of 17 patient as measued by an organ specific acute disease scale. (Antin et al., Blood 84: 1342-48, 1994).

Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Sceening Assays

Using the IL-1Ra polynucleotides of the invention, the present invention further provides methods of obtaining and identifying agents which bind to a polypeptide encoded by the ORF from a polynucleotide of the invention to a specific domain of the polypeptide encoded by a polypeptide of the invention. In detail, said method comprises the steps of:

(a) contacting an agent with an isolated IL-i Ra protein; and

(b) determining whether the agent binds to said protein or said nucleic acid.

In general, therefore, such methods for identifying compounds that bind to the IL-1Ra polynucleotide of the invention can comprise contacting a compound with a polynucleotide of the invention for a time sufficient to form a polynucleotide/compound complex, and detecting the complex, so that if a polynucleotide/compound complex is detected, a compound that binds to a polynucleotide of the invention is identified.

Likewise, in general, therefore, such methods for identifying compounds that bind to IL-1Ra polypeptide can comprise contacting a compound with a polypeptide of the invention for a time sufficient to form a polypeptide/compound complex, and detecting the complex, so that if a polypeptide/compound complex is detected, a compound that binds to IL-1Ra polynucleotide is identified.

Methods for identifying compounds that bind to a polypeptide of the invention can also comprise contacting a compound with a polypeptide of the invention in a cell for a time sufficient to form a polypeptide/compound complex, wherein the complex drives expression of a receptor gene sequence in the cell, and detecting the complex by detecting reporter gene sequence expression, so that if a polypeptide/compound complex is detected, a compound that binds a polypeptide of the invention is identified.

Compounds identified via such methods can include compounds which modulate the activity of a polypeptide of the invention (that is, increase or decrease its activity, relative to activity observed in the absence of the compound). Alternatively, compounds identified can include compounds which modulate the expression of IL-1Ra polynucleotide (that is, increase or decrease expression relative to expression levels observed in the absence of the compound). Compounds, such as compounds identified via the methods of the invention, can be tested using standard assays well known to those of skill in the art for their ability to modulate activity/expression. Also computer based drug design described below can be used to identify modulatory compounds.

The agents screened in the above assay can be, but are not limited to, peptides, carbohydrates, vitamin derivatives, small molecules or other pharmaceutical agents. The agents can be selected and screened at random or rationally selected or designed using protein modeling techniques.

For random screening, agents such as peptides, carbohydrates, pharmaceutical agents and the like are selected at random and are assayed for their ability to bind to the protein encoded by the IL-1Ra polynucleotide sequence. Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like capable of binding to a specific peptide sequence in order to generate rationally designed antipeptide peptides, for example see Hurby et al., Application of Synthetic Peptides: Antisense Peptides,” In Synthetic Peptides, A User's Guide, W. H. Freeman, NY (1992), pp. 289-307, and Kaspczak et al., Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs which rely on the same EMF for expression control. One class of DNA binding agents are agents which contain base residues which hybridize or form a triple helix formation by binding to DNA or RNA. Such agents can be based on the classic phosphodiester, ribonucleic acid backbone, or can be a variety of sulfhydryl or polymeric derivatives which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix—formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide and other DNA binding agents. Agents which bind to a protein encoded by one of the ORFs of the present invention can be formulated using known techniques to generate a pharmaceutical composition.

Computer Based Drug Design

According to one aspect of this invention, a nucleotide sequence, amino acid sequence or three-dimensional structure of the present invention can be recorded on computer readable media. A three-dimensional structure may be represented or displayed using structural coordinates of atoms of amino acids within the IL-1Ra amino acid sequences (including mutant or variant amino acid sequences), particularly amino acids involved in binding to IL-1 receptor or other receptors or IL-1 receptor accessory protein, as well as amino acids involved in other IL-1Ra functions. The crystal structure of IL-1Ra has been described in Schreuder et al. Euro. J. Biochem 227(3):838-47,1995.

As used herein, “computer readable media” or “machine readable storage medium” refers to any medium which can be read and accessed directly by a computer. The term “data storage material” refers to any material on which data can be physically stored in. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The term “machine readable data” refers to a group of one or more characters, including numbers, representing basic elements of information that can be processed by a computer. A skilled artisan can readily appreciate how any of the presently known computer readable media can be used to create a manufacture comprising a computer readable medium having recorded thereon a nucleotide sequence, amino acid sequence or structural coordinates of the present invention that can be used to render a three-dimensional structure of a polypeptide.

As used herein, “recorded” refers to a process for storing information on computer readable medium. A skilled artisan can readily adopt any of the presently known methods for recording information on computer readable medium to generate manufactures comprising the sequence or structure information of the present invention. A variety of data storage structures are available to a skilled artisan for creating a computer readable medium having recorded thereon sequence or structure information of the present invention. The choice of the data storage structure will generally be based on the means chosen to access the stored information. In addition, a variety of data processor programs and formats can be used to store the sequence or structure information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of dataprocessor structuring formats (e.g. text file or database) in order to obtain computer readable medium having recorded thereon the sequence or structure information of the present invention.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the present invention. As stated above, the computer-based systems of the present invention comprise a data storage means having stored therein sequence or structure information of the present invention and the necessary hardware means and software means for supporting and implementing a search means. As used herein, “data storage means” refers to memory which can store sequence or structure information of the present invention, or a memory access means which can access manufactures having recorded thereon the sequence or structure information of the present invention.

Input means can be implemented in a variety of ways. Machine-readable data of this invention may be inputted via the use of a modem or modems connected by a telephone line or dedicated data line. Alternatively or additionally, the input means may comprise CD-ROM drives or disk drives. In conjunction with a display terminal, a keyboard may also be used as an input device. Output means may similarly be implemented by conventional devices. By way of example, output hardware may include CRT display terminal for displaying a graphical representation of important functional residues of the invention using a computer program as described herein. Output means might also include a printer, so that hard copy output may be produced, or a disk drive to store system output for later use.

In operation, the CPU coordinates the use of the various input and output devices, coordinates data accesses from data storage means including working memory, and determines the sequence of data processing steps. A number of programs may be used to process the machine-readable data of the invention, to form or display a sequence or a three-dimensional structure or representation, or to carry out computational methods of sequence comparison or drug discovery.

For example, by providing the nucleotide sequence of IL-1Ra or a representative fragment thereof, or a nucleotide sequence at least 99.9% identical to the IL-IRa polynucleotide in computer readable form, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system is used to identify open reading frames (ORFs) within a nucleic acid sequence. Such ORFs may be protein encoding fragments and may be useful in producing commercially important proteins such as enzymes used in fermentation reactions and in the production of commercially useful metabolites.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computational methods of drug discovery may include computational evaluation of a three-dimensional structure for its ability to associate with moieties of chemical compounds. This evaluation may include performing a fitting operation between the structure or a portion thereof and one or more moieties of a chemical compound, and thereby qualitatively or quantitatively judging the proximity and/or extent of interaction between the three-dimensional structure and the chemical moiety(ies). Interaction may take place through, e.g., non-covalent interactions such as hydrogen bonding, van der Waals interactions, hydrophobic interactions and electrostatic interactions, or through covalent bonding. When the structure is displayed in a graphical three-dimensional representation on a computer screen, this allows visual inspection of the structure, as well as visual inspection of the structure's association with chemical moieties.

Specialized computer programs may be used to assist in a process of selecting chemical moieties or fragments of chemical compounds for further evaluation. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif. 4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Assembly of individual chemical moieties or fragments can be assisted by using programs including: 1. CAVEAT (P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif. 2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992). 3. HOOK (M. B. Eisen et al, “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Computer programs that assist in designing a chemical compound that potentially interacts with a three-dimensional structure as a whole or “de novo” using either an empty binding site or optionally including some portion(s) of a known modulator(s) include: 1. LUDI (H. -J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif. 2. LEGEND (Y. Nishibata et al., Tetrahedron, 47, p. 8985 (1991)). LEGEND is available from Molecular Simulations Incorporated, San Diego, Calif. 3. LeapFrog (available from Tripos Associates, St. Louis, Mo.). 4. SPROUT (V. Gillet et al, “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., N. C. Cohen et al., “Molecular Modeling Software and Methods for Medicinal Chemistry”, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida “Software For Structure-Based Drug Design”, Curr. ODin. Struct. Biology,, 4, pp. 777-781 (1994)].

Binding affinity may be tested and optimized by computational evaluation, e.g. by minimizing the energy between the bound and free states of the three-dimensional structure (e.g., a small deformation energy of binding, preferably not greater than about 10 kcal/mole and more preferably not greater than 7 kcal/mole).

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco); QUANTA/CHARMM (Molecular Simulations, Inc., San Diego, Calif.); Insight II/Discover (Molecular Simulations, Inc., San Diego, Calif); DelPhi (Molecular Simulations, Inc., San Diego, Calif.); and AMSOL (Quantum Chemistry Program Exchange, Indiana University). These programs may be implemented, for instance, using a Silicon Graphics workstation with “IMPACT” graphics. Other hardware systems and software packages will be known to those skilled in the art.

Such computational drug design may include computer-based screening of small molecule databases for chemical moieties or chemical compounds that can bind in whole, or in part, to the desired three-dimensional structure. In this screening, the quality of fit of such entities to the binding site may be judged either by shape complementarity or by estimated interaction energy [E. C. Meng et al., J. Comp. Chem., 13, pp. 505-524 (1992)].

Antisense and Triple Helix Formation

In addition, the fragments of the present invention, as broadly described, can be used to control gene expression through triple helix formation or antisense DNA or RNA, both of which methods are based on the binding of a polynucleotide sequence to DNA or RNA. Polynucleotides suitable for use in these methods are usually 20 to 40 bases in length and are designed to be complementary to a region of the gene involved in transcription (triple helix—see Lee et al., Nucl. Acids Res. 6:3073 (1979); Cooney et al., Science 15241:456 (1988); and Dervan et al., Science 251:1360 (1991)) or to the mRNA itself (antisense—Olmno, J. Neurochem. 56:560 (1991); Oligodeoxynucleotides as Antisense Inhibitors of Gene Expression, CRC Press, Boca Raton, Fla. (1988)). Triple helix—formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide.

### EXAMPLE 1

### Inhibition of IL-18 Stimulated IFN-γ Production by IL-1Ra

Human lymphocytes (PBMC) were isolated from peripheral blood of healthy volunteer donors from Stanford University Blood Center by Ficoll-Hypaque density gradient separation as described in Current Protocols in Immunology. (Ch 7, John Wily, 1998). lmnediately after isolation, the PBMC cells were washed twice with growth media (RPMI-1640 supplemented with 10% fetal bovine serum), then seeded at 3×10⁵ cells per well on a 96 well culture plate.

The PBMC cells were stimulated by adding anti-CD3 antibody (R&D Systems) to a final concentration of 0.5 μg/ml. At the time of stimulation, the wells were also treated with a 100 ng/ml human recombinant IL-18 (R&D Systems) for 36 hours at 37° C. at 5% CO₂. A portion of the wells on each plate (triplicates) were untreated to serve as a measure of background levels of IFNγ produced by stimulated PBMC cells. IL-18 treatment causes the PBMC cells to increase production of IFN-γ relative to the background levels.

In order to determine if IL-1Ra had an effect on IL-18 stimulated IFNγ production, 0.01× to 1.0× fold concentration of IL-1Ra [R&D Systems, cat. no. 280-RA] (relative to IL-18 concentration) was added in combination with 100 ng/ml IL-18 at the time of PBMC cell stimulation. After the 36 hour stimulation, the culture plates were centrifuged at 4000 rpm for 5 minutes to remove cellular debris. The concentration of IFNγ in the stimulated PBMC cell supernatants was quantified with R&D Systems Quantikine IFNγ ELISA kit according to the manufacturer's instructions.

Treatment with IL-18 resulted in an elevation of IFNγ in PBMC relative to basal levels. The relative increase in IFNγ production varied between donors but was consistently significantly increased compared to basal levels. The presence of IL-1Ra during stimulation consistently resulted in a dose dependent decrease in IL-18 induced IFNγ production. The low concentration of IL-1Ra (0.01-fold) caused a 70% decrease, while higher concentrations between 0.1-fold and 1.0-fold IL-1Ra caused complete (100%) inhibition of IL-18 induced IFNγ production.

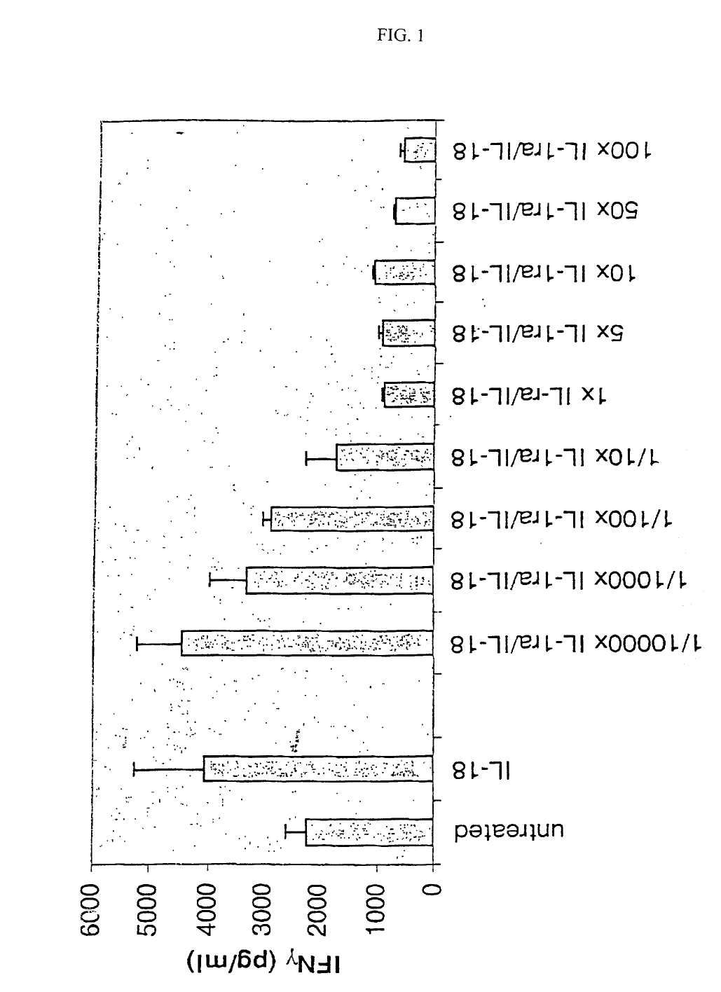

Similar results were obtained when the above experiment was repeated with a wider range of IL-1Ra concentrations (0.0001-fold to 100-fold). As indicated in FIG. 1, inhibition of IL-18 induced IFNγ production was dose dependent from 0.001-fold tol-fold. Complete inhibition of IFNγ production occurred at approximately 0.05-fold IL-1Ra.

IL-1β induced IFN-γ in a sub-set of donor samples. (Ushio et al., J. Immunol. 156: 4274-79, 1996; Tominaga et al., Int. Immunol. 12: 151-60, 2000). In those donor samples in which IFNγ production is unaffected by IL-1β treatment, IL-1Ra consistently reduces IL-18 induced IFNγ production. Although, this indicates that IL-1Ra inhibition of IL-18 induced IFN-γ production is acting independently of IL-1β, additional data in Example 6 suggests IL-1β and IL-18 may act synergistically to stimulate IFNγ production.

### EXAMPLE 2

### Inhibition of IL-18 Stimulated IFNγ Production by Blocking Antibodies

Human lymphocytes (PBMC) were isolated and stimulated with IL-18 as described in Example 1. At the time of stimulation, the PBMC cells were also treated with a blocking antibody (IL-18 receptor antibody, IL-1 receptor accessory protein antibody, IL-1 receptor type I antibody or IL-1 receptor type II antibody) in addition to 100 ng/ml of IL-18. After the 36 hour stimulation, the culture plates were centrifuged at 4000 rpm for 5 minutes to remove cellular debris. The concentration of IFNγ was measured with the Quantikine IFNγ ELISA kit as described in Example 1.

IL-18 stimulation of PBMC cells resulted in increase in IFNγ production relative to background levels. The addition of 50 μg/ml of anti-human IL-1 receptor type I monoclonal antibody (R&D Systems cat no. MAB269) significantly decreased IL-18 induced IFNγ production by 100%, returning production to that of untreated PBMC cells. Treatment with a lower concentration of IL-1 receptor type I monoclonal antibody (5 μg/ml) had no effect on IL-18 induced IFNγ production. Monoclonal antibodies to IL-1 receptor type II (R&D Systems cat. no. MAB263), at both low (5 μg/ml) and high (50 μg/ml) concentrations did not have an effect on IL-18 induced IFNγ production. The addition of 20 μg/ml of IL-18 receptor blocking monoclonal antibody (R&D Systems cat no. MAB318) completely abolished (100%) IFNγ production by PBMC cells. Similar to treatment with IL-1 receptor type I monoclonal antibody, the addition of anti-receptor accessory protein polyclonal antibody (R&D Systems cat no. AF676; 10 μg/ml) significantly decreased (100%) IL-18 induced IFNγ production to levels similar to that of unstimulated PBMC cells.

These results indicate that compounds which antagonize the action of the IL-1 receptor inhibit IL-18 activity, as measured by induction of IFNγ production. These results in combination with the data presented in Example 1 suggest that IFNγ production by IL-18 may require an IL-1 signaling component.

### EXAMPLE 3

### Inhibition of IL-12 stimulated IFNγ Production by IL-1Ra

Human lymphocytes (PBMC) were isolated as described in Example 1. Immediately after isolation, the PBMC cells were washed two times with culture media (RPMI-1640 supplemented with 10% fetal bovine serum) prior to seeding at 3×10⁵ cells/well on a 96 well culture plate. The PBMC cells were stimulated with a final concentration of 0.5 μg/ml anti-CD3 monoclonal antibody. All but 1 well of PBMC cells was incubated with 100 ng/ml of IL-12 (R&D Systems) for 36 hours at 37° C. at 5% CO₂.

To determine if IL-1Ra had an effect on IL-12 induced IFNγ production in PBMC cells, at the time of stimulation the PBMC cells were treated with 10× to 100× fold concentration of IL-1Ra [R&D Systems cat no. 280-RA] (relative to IL-12 concentration). After the 36 hour stimulation, the culture plate was centrifuged at 4000 rpm for 5 minutes to remove cellular debris. The concentration of IFNγ in the supernatant was measured with the Quantikine IFNγ ELISA kit according to the manufacturer's instructions. The stimulation with IL-12 resulted in increased production of IFNγ relative to background levels. The addition of IL-1Ra resulted in an approximately 66% decrease in IL-12 induced IFNγ production at all concentrations tested.

### EXAMPLE 4

### IL-1Ra Comparative Inhibition of IL-1β induced PGE₂ Production

Normal human dermal fibroblasts (NHDF) (Clonetics) were plated at 2×10⁴ cells per well in a 96-well plate. After 24 hours, the cells were incubated with fresh growth media (Clonetics) containing 25 μg/ml recombinant human IL-1β for 16 h. To study the inhibition of IL-1β stimulated PGE₂ release by IL-1Ra, the cells were treated with increasing concentrations of IL-1Ra (1-fold to 1000-fold) together with IL-1β. The supernatants were then collected and cell debris was removed by centrifugation. The amount of PGE₂ in the supernatants was determined by ELISA using the PGE₂ assay system (R&D Systems) according to the manufacturer's protocol. Triplicate samples were performed for each reaction. IL-1Ra inhibited IL-1β induced PGE₂ production in a dose dependent manner. Complete inhibition is seen at about 100 fold excess of IL-1Ra. Furthermore, IL-1Ra itself was unable to stimulate PGE₂ production in these cells.

In this PGE₂ assay (a classical IL-1 activity assay), it took about 100 fold molar excess of IL-1Ra to get complete inhibition of IL-1 activity. In contrast, it appears that IL-1Ra is able to exert its inhibitory activity even more potently in the IL-18 system than in the IL-1 system. (Compare Example 1). IL-1Ra appears 5000 to 10,000 times more potent for blocking IL-18 activity and IFN-γ production compared to the dose needed to effectively block IL-1β activity.

### EXAMPLE 5

### IL-18 Stimulation of IL-1β Production

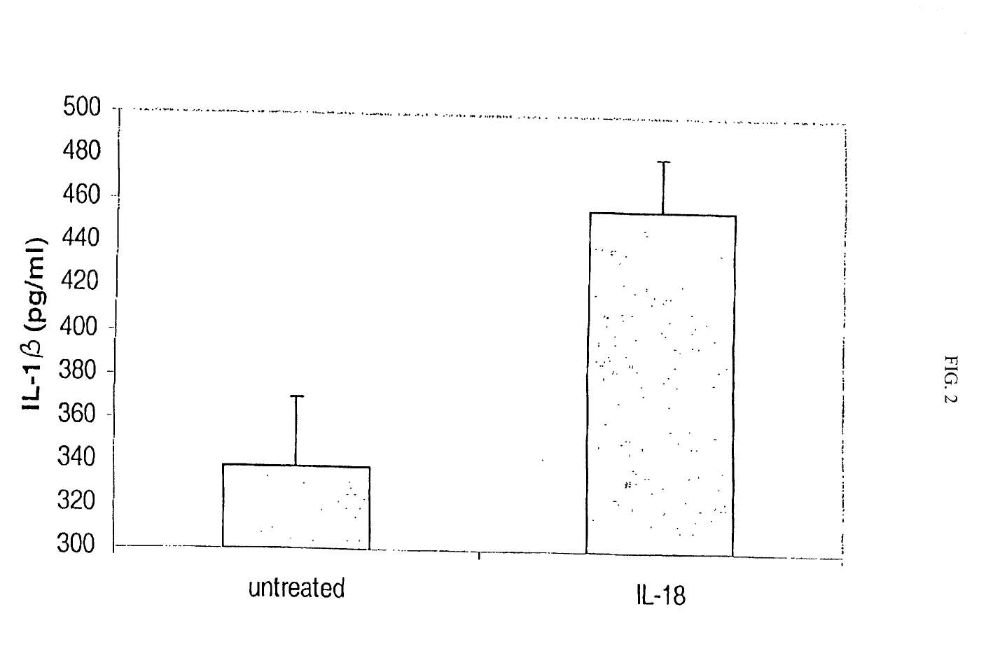

Human lymphocytes (PBMC) were isolated as described in Example 1. Immediately after isolation, the PBMC cells were washed two times with culture media (RPMI-1640 supplemented with 10% fetal bovine serum) prior to seeding at 3×10⁶ cells/ml on a 96 well culture plate. The PBMC cells were stimulated by adding anti-CD3 antibody (R&D Systems) to a final concentration of 0.5 μg/ml. At the time of stimulation, the wells were also treated with a 100 ng/ml human recombinant IL-18 (R&D Systems) for 36 hours at 37° C. at 5% CO₂. A portion of the wells on each plate (triplicates) were untreated to serve as a measure of background levels of IFNγ produced by stimulated PBMC cells. After the 36 hour stimulation, the culture plate was centrifuged at 4000 RPM for 5 minutes to remove cellular debris. The concentration of IL-1β in the supernatant was measured with the Quantikine IL-1β ELISA kit according to the manufacturer's instructions. Treatment with IL-18 significantly stimulated IL-1β production in PBMC cells as shown in FIG. 2.

### EXAMPLE 6

### Involvement of IL-1β in IFNγ Production

Human lymphocytes (PBMC) were isolated as described in Example 1. Immediately after isolation, the PBMC cells were washed two times with culture media (RPMI-1640 supplemented with 10% fetal bovine serum) prior to seeding at 3×10⁶ cells/ml on a 96 well culture plate. The PBMC cells were stimulated by adding anti-CD3 antibody (R&D Systems) to a final concentration of 0.5 μg/ml. At the time of stimulation, the wells were also treated with a 100 ng/ml human recombinant IL-13 (R&D Systems) for 36 hours at 37° C. and 5% CO₂. A portion of the wells on each plate (triplicates) were untreated to serve as a measure of background levels of IFNγ produced by stimulated PBMC cells.

After the 36 hour stimulation, the culture plate was centrifuged at 4000 RPM for 5 minutes to remove cellular debris. The concentration of IFNγ in the supernatant was measured with the Quantikine IFNγ ELISA kit according to the manufacturer's instructions. Treatment with IL-1β stimulated IFNγ production. The level of IL-1β induced IFNγ production varied between experiments but consistently was elevated between 40% to 140% as compared to IL-18 induced IFNγ production as described in Example 1. Of the 7 donor samples tested in this experiment, IL-1β stimulated IFNγ production in 5 of these samples and the negative results were not included in the data set.

The IL-1 stimulated IFNγ production was completely inhibited by 50 μg/ml of IL-1R type I antibodies as well as by IL-18R antibodies (20 μg/ml). This data indicates that IL-18 may not act independently to stimulate IFNγ production, but rather that IL-1 and IL-18 may act synergistically or are at least linked to a common biological pathway.

### EXAMPLE 7

### II-1Ra Activates Proliferation of B Cells

IL-1Ra activation of B cell proliferation was demonstrated on CA46 cells, a Burkitt's lymphoma cell line obtained from the ATCC (accession no. CRL-1648). The CA46 cells were cultured in ATCC medium (RPMI 1640 containing 2 mM L-glutamine adjusted to contain 1.5 g/L sodium bicarbonate, 4.5 g/L glucose, 10 mM HEPES, and 1.0 mM sodium pyruvate; 80%) supplemented with 20% fetal bovine serum (FBS). For each experiment, 1×10⁶ cells were preactivated with 5 μg/ml anti-IgM antibody (Irvine Scientific) for 24 hours at 37° C. in 5% CO₂. After the preincubation, 2×10⁴ cells were plated in 150 μl of ATCC media. The cells were treated with increasing concentration of IL-1Ra (R&D Systems; 0.05-500 ng/ml) and incubated for 72 hours at 37° C. in 5% CO₂. As a positive control, CA46 cells were treated with 20 ng/ml of IL-10 (R&D Systems). After the incubation, cell proliferation was measured colorimetrically using the Cell Titer assay (Promega) according to the manufacturer's instructions. The measurements were taken at O.D. 490 after a two-three hour incubation at 37° C. in 5% CO₂.

Treatment with IL-1Ra activated B-cell proliferation which resulted in a significant increase in cell proliferation compared to the untreated control. This data suggests that IL-1Ra may also have an IL-1 agonistic function in addition to its well established IL-1 antagonist function.

### EXAMPLE 8

### IL-1Ra Inhibits IL-10 Induced IgA Production

To determine if IL-1Ra affects IL-10 induced IgA production, assays on human native B cells were carried out. Human native B cells were purified from peripheral blood collected at the Stanford Blood Center according to the Mitenyi Biotec (Auburn, Calif.) purification protocols. Briefly, the samples were separated on a filcol gradient and the peripheral blood mononuclear cells were labeled with CD19 Miltenyi Biotec) for positive selection of native and memory B cells. Subsequently, depletion with CD27 (Miltenyi Biotec) was used to remove the memory B cell population.

The purified B cells were suspended at 1×10⁶ cells/ml in growth medium (Iscoves' medium supplemented with 50 μg/ml human transferrin, 5 μg/ml bovine insulin, 0.5% BSA, 5×10⁻⁵ M β-mercaptoethanol, 5% FBS and penicillin/streptomycin). The purified B cells were preactivated with 0.01% (v/v) SAC (Staphylococcus aureus strain Cowan I; Calbiochem) for 48 hours at 37° C. with 5% CO₂. Subsequently, the cells were plated at 1×10⁵ cells per well in 150 μl of growth medium containing 10 ng/ml IL-10 (R&D Systems) and various concentrations (5, 50, 500 ng/ml) of IL-1Ra (R&D Systems). After a 6 day incubation at 37° C. with 5% CO₂, the supernatant was harvested and the concentration of IgA within the supernatant was measured by an ELISA assay (Bethyl Laboratories, Montgomery, Tex.) according to the manufacturer's instructions.

Treatment with IL-10 alone caused a significant increase in B cell Ig production. The addition of IL-1Ra alone to the B cell cultures had no effect on IgA production. When the B cells were treated with both IL-10 and IL-1Ra, the IL-10 induced increase in IgA production was significantly inhibited. These results were obtained from about 80% of the experiments carried out. The slight variation was due to the use of primary B cell cultures isolated from donors.

Numerous modifications and variations of the above-described invention are expected to occur to those of skill in the art. Accordingly, only such limitations as appear in the appended claims should be placed thereon.

1. A method of treating a human suffering from an interleukin-18 (IL-18) related disorder comprising administering to said human an amount of interleukin-1 receptor antagonist polypeptide, comprising the amino acid sequence of SEQ ID NO: 1, effective to reduce IL-18 activity.

2. The method of claim 1 wherein the disorder is endotoxin-induced liver injury.

3. The method of claim 1 wherein the disorder is hepatitis.

4. The method of claim 1 wherein the disorder is hemophagocytic lymphohistiocytosis.

5. A method of claim 1 wherein the disorder is multiple sclerosis

6. The method of claim 1 wherein IL-18 activity is determined by measuring interferon-gamma (IFN-γ) activity in a sample from the human.

7. A method of treating a human suffering from an interleukin-12 (IL-12) related disorder comprising administering to said human an amount of interleukin-1 receptor antagonist polypeptide, comprising the amino acid sequence of SEQ ID NO: 1, effective to reduce IL-12 activity.

8. The method of claim 7 wherein the disorder is multiple sclerosis.

9. The method of claim 7 wherein the disorder is IL-12 induced cytotoxicity resulting from antitumor therapy.

10. The method of claim 7 wherein IL-12 activity is determined by measuring interferon-gamma (IFN-γ) activity in a sample from the human.

11. A method of claim 1 or 7 further comprising concurrently administering a second therapeutic agent for treating the disorder.

12. A method of claim 11 wherein the second therapeutic agent is an antibody to IL-18R.

13. A method of claim 11 wherein the second therapeutic agent is an antibody to IL-18.

14. A method of claim 11 wherein the second therapeutic agent is an antibody to IL-1R type I.

15. A method of claim 11 wherein the second therapeutic agent is an antibody to IL-1.

16. A method of treating inflammation comprising the steps of
(a) administering therapeutically effective amount of interleukin-1 receptor antagonist, comprising the amino acid sequence of SEQ ID NO: 1, to a human suffering from an IL-18 related disorder, and
(b) before, concurrently or after step (a), measuring IL-18 levels or activity in said human.

17. A method of treating inflammation comprising the steps of
(a) administering therapeutically effective amount of interleukin-1 receptor antagonist, comprising the amino acid sequence of SEQ ID NO: 1, to a human suffering from an IL-12 related disorder, and
(b) before, concurrently or after step (a), measuring IL-12 levels or activity in said human.

18. A method of treating inflammation comprising the steps of
(a) administering therapeutically effective amount of interleukin-1 receptor antagonist, comprising the amino acid sequence of SEQ ID NO: 1, to a human suffering from an IFN-γ related disorder, and
(b) before, concurrently or after step (a), measuring IFN-γ levels or activity in said human.

19. A method of claim 16 wherein the inflammatory condition is an endotoxin-related liver injury.

20. A method of claim 16 wherein the inflammatory condition is an hepatitis.

21. A method of claim 16 wherein the inflammatory condition is multiple sclerosis.

22. A method of claim 17 wherein the inflammatory condition is an autoimmune disease.

23. A method of claim 17 wherein the inflammatory condition is multiple sclerosis.

24. A composition comprising a dosage of IL-1Ra, or an analog thereof, that is at least 10-fold less than the dosage of IL-1Ra required to completely inhibit IL-1β induced PGE₂ production.

25. A composition of claim 24 wherein the dosage is 100-fold less than the dosage of IL-1Ra required to completely inhibit IL-1β induced PGE₂ production.

26. A composition of claim 24 wherein the dosage is 1000-fold less than the dosage of EL-1Ra required to completely inhibit IL-1β induced PGE₂ production.

27. Use of interleukin-1 receptor antagonist polypeptide comprising the amino acid sequence of SEQ ID NO: 1 in an amount effective to reduce interleukin-18 (IL-18) activity in preparation of a medicament for use in the treatment of IL-18 related disorders.

28. Use of interleukin-1 receptor antagonist polypeptide comprising the amino acid sequence of SEQ ID NO: 1 in an amount effective to reduce interleukin-12 (IL-12) activity in preparation of a medicament for use in the treatment of IL-12 related disorders.

29. A method of inhibiting B cell proliferation comprising administering an inhibitor of interleukin-1 receptor antagonist (IL-1Ra) activity to a human with elevated B cell levels or B cell activity, in an amount effective to inhibit B cell proliferation induced by IL-1Ra of SEQ ID NO: 1.

30. The method of claim 29 wherein the inhibitor is an antibody.

31. The method of claim 30 wherein the antibody is a humanized antibody.

32. The method of claim 29 wherein said human is suffering form a B cell lymphoproliferative disease.

33. The method of claim 32 wherein said human is suffering from lymphoma, leukemia or myeloma.

34. Use of an inhibitor of IL-1 receptor antagonist activity in preparation of a medicament for use in reducing B cell proliferation or activation.

35. A method of stimulating B cell proliferation comprising administering an effective amount of interleukin-1 receptor antagonist, comprising the amino acid sequence of SEQ ID NO: 1, to a human in need of higher B cell levels or activity.

37. The method of claim 36 wherein said human is suffering from a B cell deficiency.

38. The method of claim 36 wherein said human is suffering from an infection.

39. Use of IL-1 receptor antagonist polypeptide comprising the amino acid sequence of SEQ ID NO: 1 in an amount effective to stimulate proliferation of B cells in preparation of a medicament for use in stimulating B cell proliferation or activation.

40. A method of treating an IgA related autoimmune disease comprising the steps of administering a therapeutically effective amount of interleukin-1 receptor antagonist, comprising the amino acid sequence of SEQ ID NO: 1, to a human suffering from an disorder related to elevated IgA levels.

41. Use of an IL-1 receptor antagonist polypeptide comprising the amino acid sequence of SEQ ID NO: 1 in an amount effective to reduce IgA production in preparation of a medicament for use in reducing IgA production.


SEQUENCE LISTING (1 sequence)

SEQ ID NO: 1 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE